(19) United States
(12) Patent Application Publication
(10) Pub. No.: US 2011/0046720 A1
(43) Pub. Date: Feb. 24, 2011

(54) Apparatus and Method for Creating Arteriovenous Fistulas

(76) Inventors: Alon Shalev, Raanana (IL); Tiffany Lev, Modiin (IL)

Correspondence Address: The Law Office of Michael E. Kondoudis, 888 16th Street, N.W., Suite 800, Washington, DC, 20006, US

(21) Appl. No.: 12/920,184

(22) PCT Filed: Mar. 1, 2009
(86) PCT No.: PCT/IL09/00232

§ 371 Date: Nov. 8, 2010

Related U.S. Application Data
(60) Provisional application No. 61/067,759, filed on Feb. 28, 2008.

(51) Int. Cl. A61F 2/82

(52) U.S. Cl. 623/1.18; 623/1.34

(57) ABSTRACT

Apparatus (20) is provided for creating a connection between a first body lumen (114) of a subject and a second body lumen (116) of the subject. The apparatus (20) includes a first engagement portion (102) configured to engage a wall of a first body lumen (114), a second engagement portion (104) that engages a wall of a second body lumen (116), and a cylindrical connecting portion (106) connecting the first engagement portion (102) to the second engagement portion (104). The connecting portion (106) defines a channel therethrough for passage of fluid from the inner space of the lumen (114), through the flow channel, and into the inner space of the body lumen (116). The apparatus also includes a projection (200) coupled to the second engagement portion (104), and surrounds an external surface of the wall of the lumen (116). Other embodiments are also described.

### CROSS-REFERENCES TO RELATED APPLICATIONS

The present application claims priority from U.S. Provisional Patent Application No. 61/067,759 to Shalev et al., filed Feb. 28, 2008, entitled, “Apparatus and method for creating arteriovenous fistulas,” which is incorporated herein by reference.

### FIELD OF THE INVENTION

The present invention relates generally to extra-anatomical devices. More specifically, the present invention relates to an extra-anatomical device used to create and maintain an arteriovenous fistula.

### BACKGROUND OF THE INVENTION

Unless transplanted with a donated human kidney, end-stage renal disease (ESRD) patients typically undergo frequent cleansing of their blood, either by peritoneal dialysis (PD), i.e., dialysis of the abdominal space, or by hemodialysis (HD), i.e., direct dialysis of the blood.

Hemodialysis is currently used in approximately 85% of ESRD patients in the western world. Most patients visit a dialysis center three times a week, for 3-5 or more hours each week.

In 2001, over 400,000 patients received treatment for end-stage renal disease (ESRD) in the US alone, increasing by 4.2% since 2000. The number of patients with ESRD has grown consistently over the past decade, with the greatest rate of growth occurring among patients older than 75 years of age, and patients with comorbidities such as diabetes mellitus and hypertension.

Current projections indicate that the population of patients with ESRD may reach more than 2 million by 2030. The overall mortality rate has fallen by 10% since 1988, with the greatest decline among patients incident to dialysis, and an increase among patients receiving dialysis for greater than five years.

Continued growth in the number of new patients reaching ESRD, as well as improved mortality rates of ESRD patients, are both contributing to the current rise and projected epidemic of ESRD over the next 25 years. The current public health strategy of identification of patients with early kidney disease to slow their progression to ESRD, in addition to aggressive treatment strategies to minimize the morbidity and mortality of patients with ESRD, is essential toward affecting the growth and health of this population (source: Kidney International (2004) 66, S3-S7; doi:10.1111/j.1523-1755.2004.09002, which is incorporated herein by reference).

There are two common chronic means of vascular access in HD patients: (a) arteriovenous (AV) fistula, or (b) an AV graft (also termed “AV shunt”). An AV fistula is created by connecting an artery directly to a vein, usually in the forearm. This causes more blood to flow into the vein, which in turn causes the vein to grow larger and stronger, thus making repeated insertions for hemodialysis treatments easier. An AV fistula requires advance planning, because a fistula can take a year or more to mature, after its preparation.

AV Fistulae are thought to be superior to artificial AV shunts and to temporary catheters with respect to (a) their longevity, and (b) their reduced complication rates. AV fistulae are used in approximately 50% of HD patients in the US and in approximately 85%-90% of HD patients in Europe.

U.S. Pat. No. 7,059,330 to Makower describes methods, devices, and systems for a) revascularization and/or b) performing other medical procedures at vascular or non-vascular intracorporeal locations within a mammalian body. The methods generally comprise the formation of at least one extravascular passageway from a blood vessel to a vascular or non-vascular target location. In the revascularization methods the extravascular passageway is utilized for blood flow. In the medical procedure methods the extravascular passageway is utilized as a conduit for accessing or performing procedures at the vascular or non-vascular target location. Also disclosed are catheter devices and systems which are useable to form the extravascular passageways of the invention, as well as apparatus for modifying, maintaining and/or closing such extravascular passageways.

U.S. Pat. Nos. 6,231,587 and 5,830,222 to Makower describe a method and apparatus for utilizing the vascular system as a conduit to reach other vascular and extravascular locations within the body. Described are methods for revascularization wherein the extravascular passageways are formed to permit blood flow between vascular locations. Also described are methods for performing transvascular interstitial surgery (TVIS) wherein extravascular passageways are formed from a blood vessel to another vascular or non-vascular intracorporeal location. Also disclosed are devices usable for forming extravascular passageways in accordance with the invention, or for modifying, valving, maintaining or closing such passageways.

U.S. Pat. No. 6,579,311 to Makower describes devices; systems and methods for transvascular interstitial interventions, including transvascular, catheter based vascular bypass, transmyocardial revascularization, bypass grafting of blood vessels, and interstitial surgical/interventional procedures wherein a catheter is advanced translumenally through the vasculature to a desired location and an operative instrument is passed through the wall of a blood vessel and to a target location (e.g. another blood vessel, an organ, a tumor, another anatomical structure) such that one or more operative devices may be advanced to the target location to perform the desired operative or interventional procedure.

U.S. Pat. No. 6,613,081 to Kim et al. describes a plastically deformable stent for implantation within a body passage, the stent includes a plurality of cylindrical segments, and a plurality of connectors extending between adjacent segments. Each segment has an alternating pattern of curvilinear elements extending about its circumference, including first and second sets of curvilinear elements having different resistances to expansion, and preferably defining “U” shapes with alternating lengths that are connected to one another to define a substantially sinusoidal pattern. The connectors define a sinusoidal shape adapted to extend and compress axially substantially evenly when the adjacent segments are subjected to bending. The stent may be delivered on a device including an elongate member with a nose cone, an expandable member, and a proximal shoulder thereon, and an outer sheath for slidably receiving the elongate member therein. The outer sheath and/or nose cone may have perfusion holes for allowing continued perfusion of fluid during stent delivery. The device may be used in a method for implanting a stent within a curved region of a body passage, particularly for creating and/or maintaining a channel connecting a vein to an adjacent artery, preferably in the coronary system.

U.S. Pat. No. 6,616,675 to Evard et al. describes anastomotic connectors and apparatus for forming and/or maintaining connections between openings formed in anatomical structures, such as blood vessels. The apparatus is initially deployed in a first configuration which is sufficiently compact to be delivered through the lumen of a catheter or cannula. Thereafter, the device is expanded to a second configuration whereby it engages the anatomical structures and forms or maintains the desired connection between openings in the anatomical structures.

U.S. Pat. No. 5,755,775 to Trerotola et al. describes a percutaneous stent-graft for restoring blood flow between vessels. The stent-graft has a body implantable device and first and second retaining elements. Also disclosed are methods for deploying a stent-graft.

US Patent Application Publication 2002-0029079 and U.S. Pat. No. 6,432,127 to Kim et al. describe implantable connector devices which are useable to maintain fluidic connection between, or approximation of, openings formed in adjacent natural or prosthetic anatomical conduits (or adjacent openings formed in a single anatomical conduit). These connector devices generally comprise a plurality of radially expandable annular members having one or more elongate strut members extending therebetween. Initially, the device is mountable on or within a delivery catheter while in a radially compact configuration. After the delivery catheter has been inserted into the body, the device is caused to transition to a radially expanded configuration whereby it becomes implanted within the body so as to maintain the desired fluidic connection between, or the desired approximation of, the anatomical conduit(s).

U.S. Pat. No. 6,464,665 to Heuser et al. describes a catheter apparatus including an arterial catheter and a venous catheter wherein the distal ends of the catheter are insertable in an artery and a adjacent vein, respectively to a position adjacent a site for creating a fistula. The venous catheter includes a radially expandable structure adjacent the distal end which can be selectively extended outwardly to expand a portion of the wall of the vein adjacent the venous fistula site towards contact with the wall of the artery adjacent the arterial fistula site. The radially expandable structure of the venous catheter may include one or more balloons which can be inflated or a wire basket, or the vein can be expanded by injecting a fluid through an injection port into an isolated portion of the vein. The apparatus further includes a tool for creating an opening through the wall of the artery adjacent the arterial fistula site and an opening through the outwardly extending portion of the wall of the vein adjacent the venous fistula site to extend outwardly the portion of the wall of the vein towards contact with the wall of the artery. The tool may include a needle coupled to the arterial catheter adjacent the distal end of the arterial catheter, the needle being selectively switchable between a first, inactive configuration wherein the needle can be guided through the artery without causing trauma to the artery wall and a second, active configuration wherein the needle is operative to create the opening in the artery wall. The fistula between the openings maybe completed with the aid of a stent, and the proximal portion of the vein blocked with an embolization device.

The following patent and patent applications may be of interest:

PCT Publication WO 00/62711 to Rivelli

PCT Publication WO 02/02163 to Heuser

PCT Publication WO 92/00043 to Porter

U.S. Pat. No. 4,770,176 to McGreevy et al.

U.S. Pat. No. 6,039,757 to Edwards et al.

U.S. Pat. No. 6,068,638 to Makower

U.S. Pat. No. 6,068,638 to Makower

U.S. Pat. No. 6,159,225 to Makower

U.S. Pat. No. 6,261,257 to Uflacker et al.

U.S. Pat. No. 6,283,983 to Makower et al.

U.S. Pat. No. 6,409,750 to Hyodoh et al.

U.S. Pat. No. 6,585,760 to Fogarty

U.S. Pat. No. 6,746,464 to Makower

U.S. Pat. No. 6,792,979 to Konya et al.

U.S. Pat. No. 6,863,684 to Kim et al.

U.S. Pat. No. 6,974,473 to Barclay et al.

U.S. Pat. No. 7,018,401 to Hyodoh et al.

U.S. Pat. No. 7,048,014 to Hyodoh et al.

U.S. Pat. No. 7,048,014 to Hyodoh et al.

U.S. Pat. No. 7,056,325 to Makower et al.

U.S. Pat. No. 7,232,449 to Sharkawy et al.

U.S. Pat. No. 7,264,632 to Wright et al.

US Patent Application Publication 2003-0028245 to Barclay et al.

US Patent Application Publication 2004-0059280 to Makower et al.

US Patent Application Publication 2004-0133225 to Makower et al.

US Patent Application Publication 2006-0047337 to Brenneman

US Patent Application Publication 2007-0185567 to Heuser et al.

US Patent Application Publication 2007-0299384 to Faul et al.

The following articles may be of interest:

Masuda E M et al., “Stent-Graft Arteriovenous Fistula: An Endovascular Technique in Hemodialysis Access,” Journal of Endovascular Surgery: Vol. 5, No. 1, pp. 18-23 (199)

Schaub F et al., “New aspects in ultrasound-guided compression repair of postcatheterization femoral artery injuries,” Circulation 1994; 90; 1861-1865 (2004)

Trerotola S O et al., “Percutaneous creation of arteriovenous hemodialysis grafts: work in progress.” J Vasc Intery Radiol. 6(5):675-81 (1995)

Trerotola S O et al., “Comparison of Gianturco Z stents and Wallstents in a hemodialysis access graft animal model.” J Vasc Intery Radiol. 1995 May-June; 6(3):387-96 (1995)

### SUMMARY OF THE PRESENT INVENTION

In some embodiments of the present invention, a method and apparatus are provided for performing a percutaneous transluminal generation of an extra-anatomical fistula between two body lumens of a subject. Typically, the fistula apparatus is transluminally implanted within the subject during an outpatient vascular microsurgery procedure. Typically, the apparatus comprises a stent further comprising a first engagement portion, a second engagement portion, and a substantially cylindrical connecting portion. In some embodiments, the apparatus creates a fistula for draining blood from a high-pressure vessel to a lower pressure vessel, e.g., from a high-pressure vein to a low-pressure vein or from an artery to a vein. In some embodiments, the apparatus creates an arteriovenous fistula for draining blood from an artery to a vein.

Typically, the apparatus is transluminally inserted by advancing the apparatus in a compressed configuration thereof through the first body lumen and toward a site designated for implantation of the fistula apparatus. The apparatus is advanced through the first opening created in the first lumen, through a space defined by the first and second lumens, and toward the second opening formed in the second lumen. Following the advancement, the first engagement portion is disposed in proximity with the first opening formed in the first body lumen. Additionally, following the advancement of the apparatus, a channel is formed in the space defined by the first and second body lumens, and the connecting portion is disposed in the channel in order to connect the first body lumen to the second body lumen.

In some embodiments, the first engagement portion engages at least a portion of an inner lumen of a first body lumen of the subject, in proximity to a first opening created therein, and the second engagement portion comprises a projection which engages at least a portion of an external wall of a second body lumen of the subject, in proximity to a second opening formed therein. Typically, the projection engages the external wall of the second body lumen by being wrapped therearound. For embodiments in which the first body lumen includes a vein and the second body lumen includes an artery, draining the blood from the artery to the vein facilitates enlargement and strengthening of the draining vein for repeated insertions therein of hemodialysis equipment, by way of illustration and not limitation.

In some embodiments, the first engagement portion engages the first body lumen from an external portion thereof. In some embodiments, the first engagement portion is shaped to define a flared end which is configured to expand radially from the first opening created in the first body lumen. In some embodiments, the first engagement portion is shaped to define at least one tubular element, e.g., two tubular elements, which is configured to be disposed within the first lumen substantially perpendicularly with respect to an axis defined by the connecting portion.

In some embodiments, the second engagement portion comprises at least one, e.g., two, substantially tubular element, e.g., a stent, which is configured to be disposed within the second body lumen, e.g., the high-pressure vessel. Typically, the tubular portion is coupled to the connecting portion at a junction defining a circumference thereof. The tubular portion is coupled to the connecting portion along an arc of the circumference which subtends an angle that is less than 180 degrees, e.g., less than 90 degrees. In some embodiments, the arc subtends an angle that is between 30 degrees and 90 degrees.

Once the apparatus is deployed within the body of the subject, the tubular element is configured to be disposed within the second lumen substantially perpendicularly with respect to the axis defined by the connecting portion.

In some embodiments, the apparatus is shaped to define at least one aperture, (e.g., a slit, an elliptical aperture, or a general discontinuity of the body portions defining the apparatus), at the junction between the connection portion and the first engagement portion. For embodiments in which the apparatus comprises a stent comprising a plurality of strands that are interconnected: (1) the stands of the stent define a plurality of stent windows, and (2) the aperture of the apparatus defines an area that is typically larger than the size of an individual window created by the strands of the stent. In some embodiments, the aperture is shaped to define a lateral aperture. In some embodiments, the aperture is shaped to define a longitudinal aperture. Alternatively or additionally, the apparatus is shaped to define the aperture, at the junction between the connection portion and the second engagement portion. Typically, the aperture enables the apparatus to be deployed in such a way that facilitates unobstructed flow of fluid within the body lumen in contact with the first and/or second engagement portions. Once deployed, the engagement portions of the apparatus define (1) respective channels having a diameter substantially equal to the respective diameters of the first and second body lumens, and (2) proximal and distal (i.e., with respect to the flow of blood in the lumen) openings having a diameter substantially equal to the respective diameters of the first and second body lumens. These openings are facilitated by the apertures defined by the apparatus and minimize the formation of fibrosis within respective transverse planes of the first and second portions that are substantially perpendicular with respect to the flow of blood within the first and second body lumens.

There is therefore provided, in accordance with an embodiment of the invention apparatus including:

at least one first engagement portion configured to engage the wall of the first body lumen;

at least one second engagement portion configured to engage the wall of the second body lumen;

a substantially cylindrical connecting portion connecting the at least one first engagement portion to the at least one second engagement portion, the connecting portion:
being configured to define a flow channel therethrough for passage of fluid from the inner space of the first body lumen, through the first opening, through the flow channel, through the second opening, and into the inner space of the second body lumen, and
being shaped to define a longitudinal connecting portion axis thereof; and
at least one projection coupled to the second engagement portion, the at least one projection configured to surround at least a portion of an external surface of the wall of the second body lumen.

In an embodiment:
the first body lumen includes an artery,
the second body lumen includes a vein,
the first engagement portion is configured to engage the artery, and
the second engagement portion is configured to engage the vein.

In an embodiment:
the first body lumen includes a first vein,
   the second body lumen includes a second vein,
the first engagement portion is configured to engage the first vein, and
the second engagement portion is configured to engage the second vein.

In an embodiment, the at least one projection is shaped to define a helical configuration.

In an embodiment, the apparatus is configured to be transluminally implanted within an implantation site of the subject.

In an embodiment, the at least one projection includes a bifurcated projection being bifurcated to a first subprojection and a second subprojection, and:

the first subprojection is configured to surround at least in part a lateral surface of the second body lumen proximally adjacent to the second opening, and

the second subprojection is configured to surround at least in part the lateral surface, distally adjacent to the second opening.

In an embodiment, the cylindrical connecting portion includes a self-expanding stent.

In an embodiment, the cylindrical connecting portion includes a mesh.

In an embodiment, the apparatus includes a self-expanding stent.

In an embodiment, the cylindrical connecting portion includes a shape-memory alloy.

In an embodiment, the cylindrical connecting portion is configured to assume a predetermined shape.

In an embodiment, the apparatus includes a superelastic alloy.

In an embodiment, the cylindrical connecting portion includes a superelastic alloy.

In an embodiment, the cylindrical connecting portion includes a plurality of filaments, the plurality of filaments configured to be helically wound and to define an open weave configuration.

In an embodiment, the plurality of filaments include the superelastic alloy.

In an embodiment, the at least one projection is configured to surround at least in part the external surface of the wall of the second body lumen.

In an embodiment, the at least one projection is configured to surround between 90 and 270 degrees of the wall of the second body lumen.

In an embodiment, the projection is configured to surround substantially 360 degrees of the wall of the second body lumen.

In an embodiment, during a first period, the apparatus is configured to be disposed in a radially collapsed configuration, and during a second period the apparatus is configured to be disposed in a radially expanded configuration.

In an embodiment, during the second period:
the first engagement portion is configured to engage the first lumen from a site within the inner space of the first body lumen, and
the second engagement portion is configured to engage the second body lumen at a site along an external surface of the wall of the second body lumen.

In an embodiment, during the second period, the first engagement portion is configured to engage the first lumen at a site along an external surface of the wall of the first body lumen first body lumen adjacently to the first opening.

In an embodiment, during the first period, the apparatus is configured to be transluminally advanced toward a space between the first body lumen and the second body lumen.

In an embodiment:
the first engagement portion includes a first radiopaque marker configured to indicate an orientation of the first engagement portion with respect to the connecting portion, and

the second engagement portion includes a second radiopaque marker configured to indicate an orientation of the second engagement portion with respect to the connecting portion.

In an embodiment:
the first engagement portion includes a first radiopaque marker configured to indicate position of the first engagement portion, and
the second engagement portion includes a second radiopaque marker configured to indicate a position of the second engagement portion.

In an embodiment, the at least one projection includes a shape-memory alloy, and during the second period, the at least one projection is configured to assume a predetermined configuration and surround the external wall of the second lumen.

In an embodiment, during the second period, the projection is configured to surround between 90 and 360 degrees of the external wall of the second lumen.

In an embodiment, the projection is configured to assume a helical configuration and helically surround the external wall of the second lumen.

In an embodiment, during the second period, the first engagement portion is shaped to define a flared configuration having a generally oval transverse cross-section.

In an embodiment, during the second period, the first engagement portion is configured to flare radially from the first opening along an axis substantially perpendicular to the longitudinal connecting portion axis.

In an embodiment, during the second period, the oval transverse section has a major axis having a length of between 3 mm and 6 mm.

In an embodiment, during the second period, the cylindrical connecting portion has a diameter of between 2 mm and 5 mm.

In an embodiment, further including a biodegradable film surrounding at least a portion of the connecting portion, the covering film configured to define a fluid-impervious lumen of the connecting portion.

In an embodiment, the apparatus is configured to be transluminally implanted within an implantation site, and at least 90 percent of the covering film is configured to degrade within less than 30 days following the implantation.

In an embodiment, the film includes at least one polymer selected from the group consisting of: PLGA, collagen, and gelatin.

There is also provided, in accordance with an embodiment of the invention, apparatus, including:

at least one first engagement portion configured to engage the wall of first body lumen;

at least one second engagement portion configured to engage the wall of the second body lumen; and

a substantially cylindrical connecting portion connecting the at least one first engagement portion to the at least one second engagement portion, the connecting portion:

being configured to define a proximal end, a distal end, and a flow channel through the connecting portion for passage of fluid from the inner space of the first body lumen, through the first opening, through the flow channel, through the second opening, and into the inner space of the second body lumen,

being shaped to define a longitudinal connecting portion axis thereof, and

being coupled at the distal end thereof to the at least one second engagement portion along an arc of a circumference of the distal end of the connecting portion, the arc subtending an angle less than 180 degrees.

In an embodiment, the arc of the circumference of the distal end of the connecting portion subtends an angle between 30 degrees and 90 degrees.

In an embodiment:
the first body lumen includes an artery,
the second body lumen includes a vein,
the first engagement portion is configured to engage the artery, and
the second engagement portion is configured to engage the vein.

In an embodiment:
the first body lumen includes a first vein,
the second body lumen includes a second vein,
the first engagement portion is configured to engage the first vein, and
the second engagement portion is configured to engage the second vein.

In an embodiment, the first engagement portion is configured to engage the first lumen from a site within the inner space of the first body lumen.

In an embodiment, the apparatus is configured to be transluminally implanted within an implantation site of the subject.

In an embodiment, the cylindrical connecting portion has a diameter between 2 mm and 5 mm.

In an embodiment, the cylindrical connecting portion has a length between 3 mm and 20 mm.

In an embodiment, the cylindrical connecting portion includes a self-expanding stent.

In an embodiment, the cylindrical connecting portion includes a mesh.

In an embodiment, the apparatus includes a self-expanding stent.

In an embodiment, the cylindrical connecting portion includes a shape-memory alloy.

In an embodiment, the cylindrical connecting portion is configured to assume a predetermined shape.

In an embodiment, the apparatus includes a superelastic alloy.

In an embodiment, the cylindrical connecting portion includes a superelastic alloy.

In an embodiment, the cylindrical connecting portion includes a plurality of filaments, the plurality of filaments configured to be helically wound and to define an open weave configuration.

In an embodiment, the plurality of filaments include the superelastic alloy.

In an embodiment, further including a biodegradable film surrounding at least a portion of the connecting portion, the covering film configured to define a fluid-impervious lumen of the connecting portion.

In an embodiment, the apparatus is configured to be transluminally implanted within an implantation site, and at least 90 percent of the covering film is configured to degrade within less than 30 days following the implantation.

In an embodiment, the film includes at least one polymer selected from the group consisting of: PLGA, collagen, and gelatin.

In an embodiment, during a first period, the apparatus is configured to be disposed in a radially collapsed configuration, and during a second period the apparatus is configured to be disposed in a radially expanded configuration.

In an embodiment, during the second period, the first engagement portion is configured to engage the first lumen at a site along an external surface of the wall of the first body lumen first body lumen.

In an embodiment, during the second period:
the first engagement portion is configured to engage the first lumen at a site along an external surface of the wall of the first body lumen, and
the second engagement portion is configured to engage the second body lumen at a site along an external surface of the wall of the second body lumen.

In an embodiment, during the second period:
the first engagement portion is configured to engage the first lumen from a site within the inner space of the first body lumen, and
the second engagement portion is configured to engage the second body lumen at a site along an external surface of the wall of the second body lumen.

In an embodiment, during the first period, the apparatus is configured to be transluminally advanced toward a space between the first body lumen and the second body lumen.

In an embodiment:
the first engagement portion includes a first radiopaque marker configured to indicate an orientation of the first engagement portion with respect to the connecting portion, and
the second engagement portion includes a second radiopaque marker configured to indicate an orientation of the second engagement portion with respect to the connecting portion.

In an embodiment:
the first engagement portion includes a first radiopaque marker configured to indicate a position of the first engagement portion, and
the second engagement portion includes a second radiopaque marker configured to indicate a position of the second engagement portion.

In an embodiment, during the second period, the first engagement portion is shaped to define a flared configuration having a generally oval transverse cross-section.

In an embodiment, during the second period, the first engagement portion is configured to engage an internal surface of the wall of the first body lumen.

In an embodiment, during the second period, the first engagement portion is configured to flare radially from the first opening along an axis substantially perpendicular to the longitudinal connecting portion axis.

In an embodiment, during the second period, the first engagement portion is configured to engage an external surface of the wall of the first body lumen.

In an embodiment, during the second period, the oval transverse section has a major axis having a length of between 3 mm and 6 mm.

In an embodiment, the first engagement portion includes at least one substantially tubular element, the tubular element being coupled to the proximal end of the connecting portion along an arc of a circumference of the proximal end of the connecting portion, the arc subtending an angle less than 180 degrees.

In an embodiment, during the second period, the tubular element is shaped to define a length of between 2 mm and 5 mm.

In an embodiment, during the second period, the tubular element is shaped to define a diameter of between 2 mm and 6 mm.

In an embodiment, the arc of the circumference of the proximal end of the connecting portion subtends an angle between 30 degrees and 90 degrees.

In an embodiment, during the second period:

the tubular element is configured to be disposed within the first body lumen in proximity to the first opening formed therein, and

the tubular element aligns substantially in parallel with a longitudinal axis of the first body lumen.

In an embodiment, the at least one tubular element includes a stent.

In an embodiment, during the second period, the first engagement portion is configured to facilitate substantially unobstructed flow of fluid within the inner space of the first body lumen.

In an embodiment, during the second period, the tubular element is configured to be disposed substantially perpendicularly with respect to the longitudinal connecting portion axis of the connecting portion.

In an embodiment, during the second period, the tubular element is configured to be disposed at and angle of between 30 degrees and 90 degrees with respect to the longitudinal connecting portion axis of the connecting portion.

In an embodiment, the first engagement portion includes a first tubular element and a second tubular element, each tubular element being coupled to the connecting portion along first and second respective arcs of the circumference of the proximal end of the connecting portion, the first and second arcs subtending respective first and second angles that are each less than 180 degrees.

In an embodiment, the first and second angles are each between 90 degrees and 120 degrees.

In an embodiment, the first and second angles are each between 40 degrees and 90 degrees.

In an embodiment, the first and second tubular elements include a stent.

In an embodiment, during the second period:
the first tubular element is configured to be disposed within the first body lumen in proximity to a first side of the first opening formed therein,
the second tubular element is configured to be disposed within the first body lumen in proximity to a second side of the first opening formed therein, and
the first and second tubular elements are each aligned substantially in parallel with a longitudinal axis of the first body lumen.

In an embodiment, during the second period, the first and second tubular elements of the first engagement portion are configured to facilitate substantially unobstructed flow of fluid within the inner space of the first body lumen.

In an embodiment, during the second period, the first and second tubular elements are configured to be disposed substantially perpendicularly with respect to the longitudinal connecting portion axis of the connecting portion.

In an embodiment, during the second period, the first and second tubular elements are configured to be disposed at an angle of between 30 degrees and 90 degrees with respect to the longitudinal connecting portion axis of the connecting portion.

In an embodiment, the second engagement portion includes at least one substantially tubular element, and during the second period:

the tubular element is configured to be disposed within the second body lumen in proximity to the second opening formed therein, and

the tubular element aligns substantially in parallel with a longitudinal axis of the second body lumen.

In an embodiment, during the second period, the second engagement portion is configured to facilitate substantially unobstructed flow of fluid within the inner space of the second body lumen.

In an embodiment, the tubular element includes a stent.

In an embodiment, during the second period, the tubular element is configured to be disposed substantially perpendicularly with respect to the longitudinal connecting portion axis of the connecting portion.

In an embodiment, during the second period, the tubular element is configured to be disposed at an angle of between 30 degrees and 90 degrees with respect to the longitudinal connecting portion axis of the connecting portion.

In an embodiment, the second engagement portion includes a first tubular element and a second tubular element, each tubular element being coupled to the connecting portion along first and second respective arcs of the circumference of the distal end of the connecting portion, the first and second arcs subtending respective first and second angles that are each less than 180 degrees.

In an embodiment, the first and second angles are each between 90 degrees and 120 degrees.

In an embodiment, the first and second angles are each between 40 degrees and 90 degrees.

In an embodiment, the first and second tubular elements include a stent.

In an embodiment, during the second period:
the first tubular element is configured to be disposed within the second body lumen proximally adjacent to the second opening formed therein,
the second tubular element is configured to be disposed within the second body lumen distally adjacent to the second opening formed therein, and

the first and second tubular elements each aligned substantially in parallel with a longitudinal axis of the second body lumen.

In an embodiment, during the second period, the first and second tubular elements of the second engagement portion are configured to facilitate substantially unobstructed flow of fluid within the inner space of the second body lumen.

In an embodiment, during the second period, the first and second tubular elements are configured to be disposed substantially perpendicularly with respect to the longitudinal connecting portion axis of the connecting portion.

In an embodiment, during the second period, the first and second tubular elements are configured to be disposed at an angle of between 30 degrees and 90 degrees with respect to the longitudinal connecting portion axis of the connecting portion.

In an embodiment, during a first period, the first engagement portion, the second engagement portion, and the connection portion are collectively shaped to define a single substantially cylindrical tube.

In an embodiment, the cylindrical tube includes a self-expanding stent.

In an embodiment, the cylindrical tube includes a mesh.

In an embodiment, the cylindrical tube includes a superelastic alloy.

In an embodiment, the cylindrical tube includes a plurality of filaments, the plurality of filaments configured to be helically wound and to define an open weave configuration.

In an embodiment, the plurality of filaments include the superelastic alloy.

In an embodiment, the cylindrical tube is shaped to define at least one lateral aperture in a vicinity of a junction between the connecting portion and the second engagement portion.

In an embodiment, the lateral aperture is shaped to define a slit.

In an embodiment, the cylindrical portion in an expanded state thereof has a diameter thereof, and the aperture has a characteristic diameter thereof being between one half and two times the diameter of the cylindrical portion.

In an embodiment, the connecting portion has a circumference at the vicinity, and the lateral aperture is shaped to define an arc subtending an angle of up to 300 degrees of the circumference at the vicinity.

In an embodiment, the connecting portion has a circumference at the vicinity, and the lateral aperture is shaped to define an arc subtending an angle of up to 180 degrees of the circumference at the vicinity.

In an embodiment, the connecting portion has a circumference at the vicinity, and the lateral aperture is shaped to define an arc subtending an angle of between 180 degrees and 300 degrees of the circumference at the vicinity.

In an embodiment, the cylindrical tube is shaped to define at least one lateral aperture in a vicinity of a junction between the cylindrical portion and the first engagement portion.

In an embodiment, the lateral aperture is shaped to define a slit.

In an embodiment, the cylindrical portion in an expanded state thereof has a diameter thereof, and the aperture has a characteristic diameter thereof being between one half and two times the diameter of the cylindrical portion.

In an embodiment, the connecting portion has a circumference at the vicinity, and the lateral aperture is shaped to define an arc subtending an angle of up to 300 degrees of the circumference at the vicinity.

In an embodiment, the connecting portion has a circumference at the vicinity, and the lateral aperture is shaped to define an arc subtending an angle of up to 180 degrees of the circumference at the vicinity.

In an embodiment, the connecting portion has a circumference at the vicinity, and the lateral aperture is shaped to define an arc subtending an angle of between 180 degrees and 300 degrees of the circumference at the vicinity.

In an embodiment, the cylindrical tube is shaped to define:

at least one first aperture in a first vicinity of a junction between the connecting portion and the first engagement portion, and

at least one second aperture in a second vicinity of a junction between the connecting portion and the second engagement portion.

In an embodiment, the cylindrical portion in an expanded state thereof has a diameter thereof, and the first and second apertures each have a respective characteristic diameter thereof being between one half and two times the diameter of the cylindrical portion.

In an embodiment, the first aperture is shaped to define at least one first longitudinal aperture.

In an embodiment, the first aperture is shaped to define at least one first longitudinal slit.

In an embodiment, the first and second apertures are shaped to define respective first and second lateral apertures.

In an embodiment, the first and second lateral apertures are shaped to define respective first and second lateral slits.

In an embodiment,:
the cylindrical connecting portion has a first circumference at the first vicinity and a second circumference at the second vicinity,
the first lateral aperture is shaped to define an arc subtending an angle of up to 300 degrees of the first circumference at the first vicinity, and
the second lateral aperture is shaped to define an arc subtending an angle of up to 300 degrees of the second circumference at the second vicinity.

In an embodiment, wherein:
the first lateral aperture is shaped to define an arc subtending an angle of up to 180 degrees of the first circumference at the first vicinity, and
the second lateral aperture is shaped to define an arc subtending an angle of up to 180 degrees of the second circumference at the second vicinity.

In an embodiment:
the first lateral aperture is shaped to define an arc subtending an angle of between 180 degrees and 300 degrees of the first circumference at the first vicinity, and
the second lateral aperture is shaped to define an arc subtending an angle of between 180 degrees and 300 degrees of the second circumference at the second vicinity.

In an embodiment, the first engagement portion is shaped to define at least one longitudinal slit extending from a proximal portion of the first engagement portion to a junction between the first engagement portion and the connecting portion.

In an embodiment, the first engagement portion is shaped to define two longitudinal slits extending from the proximal portion of the first engagement portion to the junction, and the slits define first and second distinct portions of the first engagement portion.

In an embodiment:
the first and second portions include a shape-memory alloy,
during a first period, the first and second portions of the first engagement portion are configured to align in parallel with the longitudinal connecting portion axis, and
during a second period, the first and second portions of the first engagement portion change shape to assume respective first and second substantially tubular elements.

In an embodiment, during the second period, the first and second tubular elements are configured to bend away from the connecting portion along an axis substantially perpendicular to the longitudinal connecting portion axis.

There is additionally provided, in accordance with an embodiment of the invention, method including:

creating a first opening in the first body lumen;

creating a second opening in the second body lumen;

widening a channel between the first and second openings;

advancing into the channel at least a portion of a substantially tubular element, the tubular element having a proximal portion configured to engage the first lumen and a distal portion configured to engage the second lumen; and

surrounding a portion of an external wall of the second lumen by the distal portion of the tubular element.

In an embodiment, creating the fistula includes transluminally creating the fistula.

In an embodiment, further including surrounding a portion of an external wall of the first lumen by a proximal portion of the tubular element.

In an embodiment, surrounding the portion of the external wall of the second lumen includes allowing a portion of the distal portion of the tubular element to expand around the second body lumen.

In an embodiment:
creating the first opening in the first body lumen includes creating the first opening in an artery of the subject,
creating the second opening in the second body lumen includes creating the second opening in a vein of the subject, and
widening the channel includes creating an arteriovenous channel.

In an embodiment:
creating the first opening in the first body lumen includes creating the first opening in a first vein of the subject, and
creating the second opening in the second body lumen includes creating the second opening in a second vein of the subject.

In an embodiment:
creating the first opening in the first lumen includes advancing through the first lumen a tissue-penetrating member having a lumen extending therethrough,
widening the channel includes advancing the tissue-penetrating member beyond the first opening and toward the second body lumen, and
creating the second opening in the second lumen includes advancing the tissue-penetrating member through the second body lumen.

In an embodiment, further including monitoring, using ultrasound guidance, the creating of the first opening, the creating of the second opening, and the widening of the channel.

In an embodiment, further including:
(a) inserting a guidewire through the lumen of the tissue-penetrating member and into the first body lumen;
(b) removing the tissue penetrating member; and
(c) sliding the substantially tubular element in a constricted configuration thereof around the guidewire and into the channel; and
(d) deploying the tubular element within the channel.

In an embodiment, further including monitoring steps (a) through (d) using ultrasound guidance.

In an embodiment, the tubular element includes a shape-memory alloy, and surrounding the portion of the external wall of the second lumen includes allowing the shape-memory alloy to expand to a predetermined shape and surround the second lumen.

In an embodiment, deploying the tubular element within the channel includes passively allowing the stent to expand inside the channel.

In an embodiment, advancing into the channel the portion of, the substantially tubular element includes advancing a tubular element surrounded by a biodegradable film configured to degrade within less than 90 days following the advancing.

In an embodiment, advancing into the channel the portion of the substantially tubular element includes advancing a tubular element surrounded by a biodegradable film configured to degrade within less than 30 days following the advancing.

There is additionally provided, in accordance with an embodiment of the invention, a stent, including:
a proximal portion;
a distal portion; and
a connecting portion disposed intermediate the proximal and distal portions, the connecting portion having:
a first circumference at a vicinity of a junction between the connecting portion and the proximal portion, and
a second circumference at a vicinity of a junction between the connecting portion and the distal portion, and

the stent in an expanded state thereof having a diameter thereof, and shaped to define:
at least a first aperture, at least a portion thereof being at the junction between the connecting portion and the proximal portion, a characteristic diameter of the first aperture being between one half and two times the diameter of the stent, and
at least a second aperture, at least a portion thereof being at the junction between the connecting portion and the distal portion, a characteristic diameter of the second aperture being between one half and two times the diameter of the stent.

In an embodiment, the first circumference is equal to the second circumference.

In an embodiment, the first circumference is not equal to the second circumference.

In an embodiment, the first aperture is shaped to define at least one first longitudinal aperture.

In an embodiment, the first aperture is shaped to define at least one first longitudinal slit.

In an embodiment, the first and second apertures are shaped to define respective first and second lateral apertures.

In an embodiment, the first and second lateral apertures are shaped to define respective first and second lateral slits.

In an embodiment, the first aperture is shaped to define an arc subtending an angle of up to 300 degrees of the first circumference, and the second aperture is shaped to define an arc subtending an angle of up to 300 degrees of the second circumference.

In an embodiment, the first aperture is shaped to define an arc subtending an angle of up to 180 degrees of the first circumference, and the second aperture is shaped to define an arc subtending an angle of up to 180 degrees of the second circumference.

In an embodiment, the first aperture is shaped to define an arc subtending an angle of between 180 degrees and 300 degrees of the first circumference, and the second aperture is shaped to define an arc subtending an angle of between 180 degrees and 300 degrees of the second circumference.

In an embodiment, the second engagement portion is shaped to define at least one longitudinal slit extending from a distal portion of the second engagement portion to the junction between the connecting portion and the second engagement portion.

In an embodiment, the second engagement portion is shaped to define two longitudinal slits extending from the distal portion of the second engagement portion to the junction, and the slits define first and second distinct portions of the second engagement portion.

In an embodiment:
the first and second portions include a shape-memory alloy,
during a first period, the first and second portions of the second engagement portion are configured to align in parallel with the longitudinal connecting portion axis, and
during a second period, the first and second portions of the second engagement portion change shape to assume respective first and second substantially tubular elements.

In an embodiment, during the second period, the first and second tubular elements are configured to bend away from the connecting portion along an axis substantially perpendicular to the longitudinal connecting portion axis.

In an embodiment, the first engagement portion is shaped to define at least one longitudinal slit extending from a proximal portion of the first engagement portion to a junction between the first engagement portion and the connecting portion.

In an embodiment, the first engagement portion is shaped to define two longitudinal slits extending from the proximal portion of the first engagement portion to the junction, and the slits define first and second distinct portions of the first engagement portion.

In an embodiment:
the first and second portions include a shape-memory alloy,
during a first period, the first and second portions of the first engagement portion are configured to align in parallel with the longitudinal connecting portion axis, and
during a second period, the first and second portions of the first engagement portion assume respective first and second substantially tubular elements.

In an embodiment, during the second period, the first and second tubular elements are configured to bend away from the connecting portion along an axis substantially perpendicular to the longitudinal connecting portion axis.

In an embodiment, the stent includes a shape-memory alloy.

In an embodiment, the stent is configured to assume a predetermined shape.

In an embodiment, the proximal portion is configured to engage a first body lumen and the distal portion is configured to engage a second body lumen.

In an embodiment, the proximal and distal portions are configured to facilitate substantially unobstructed flow of fluid within the first and second body lumens, respectively.

In an embodiment:
the first body lumen includes a high-pressure lumen,
the second body lumen includes a low-pressure lumen,
the first engagement portion is configured to engage the high-pressure lumen, and
the second engagement portion is configured to engage the low-pressure lumen.

In an embodiment:
the first body lumen includes an artery,
the second body lumen includes a vein,
the proximal portion is configured to engage the artery, and
the distal portion is configured to engage the vein.

In an embodiment:
the first body lumen includes a first vein,
the second body lumen includes a second vein,
the proximal portion is configured to engage the first vein, and
the distal portion is configured to engage the second vein.

In an embodiment, during a first period, the stent is configured to be disposed in a radially collapsed configuration, and during a second period the stent is configured to be disposed in a radially expanded configuration.

In an embodiment, during the first period, the stent is configured to be positioned at least in part in a space between a first body lumen and a second body lumen.

In an embodiment, during the first period, the stent is configured to be positioned at least in part in an interstitial space between the first body lumen and the second body lumen.

In an embodiment, during the second period:
the proximal portion is configured to engage the first lumen from a site within the first body lumen, and
the distal portion is configured to engage the first lumen from a site within the second body lumen.

In an embodiment, during the first period, the stent is configured to be transluminally advanced toward the space between the first body lumen and the second body lumen.

In an embodiment:
the proximal portion includes a proximal radiopaque marker configured to indicate an orientation of the proximal portion with respect to the connecting portion, and
the distal portion includes a distal radiopaque marker configured to indicate an orientation of the distal portion with respect to the connecting portion.

In an embodiment:
the proximal portion includes a proximal radiopaque marker configured to indicate a position of the proximal portion, and
the distal portion includes a distal radiopaque marker configured to indicate a position.

There is additionally provided, in accordance with an embodiment of the invention, method including:

creating a first opening in the first body lumen;

creating a second opening in the second body lumen;

widening a channel between the first and second openings;

advancing into the channel at least a portion of a substantially tubular element, the tubular element:
having a proximal portion configured to engage the first lumen,
having a distal portion configured to engage the second lumen,
having a connecting portion disposed intermediate the proximal and distal portions,
being shaped to define at least a first lateral aperture at a junction between the connecting portion and the proximal portion, and
being shaped to define at least a second lateral aperture at a junction between the connecting portion and the distal portion;
inserting the distal portion into the second body lumen; and

expanding the distal portion within the second body lumen.

There is additionally provided, in accordance with an embodiment of the invention, a stent, including:

a proximal portion;

a distal portion; and

a coupling junction disposed intermediate the proximal and distal portions, the junction having a circumference thereof, and

the stent in an expanded state thereof having a diameter thereof, and shaped to define:

an aperture at the junction, a characteristic diameter of the aperture being between one half and two times the diameter of the stent, the aperture subtending an angle between 180 degrees and 300 degrees of the circumference at the junction.

The present invention will be more fully understood from the following detailed description of embodiments thereof, taken together with the drawings, in which:

### BRIEF DESCRIPTION OF THE DRAWINGS

FIGS. 1A and 1B are schematic illustrations of an apparatus for creating a fistula, in accordance with an embodiment of the present invention;

FIGS. 2A and 2B are schematic illustrations of the apparatus for creating a fistula, in accordance with another embodiment of the present invention;

FIGS. 3A and 3B are schematic illustrations of the apparatus for creating a fistula, in accordance with yet another embodiment of the present invention;

FIGS. 4A and 4B are schematic illustrations of the apparatus for creating a fistula of FIGS. 3A and 3B, in accordance with still another embodiment of the present invention;

FIGS. 5 and 6 are schematic illustrations of the delivery of the apparatus for creating a fistula in accordance with respective embodiments of the present invention;

FIGS. 7A and 7B are schematic illustrations of the apparatus for creating a fistula, in accordance with still yet another embodiment of the present invention;

FIGS. 7C and 7D are schematic illustrations of the delivery and deployment of the apparatus of FIGS. 7A and 7B, in accordance with an embodiment of the present invention;

FIG. 7E is a schematic illustration of the deployment of the apparatus of FIG. 7A, in accordance with an embodiment of the present invention, and

FIGS. 8-13 are schematic illustrations of a distal portion of the apparatus for creating a fistula, in accordance with respective embodiments of the present invention.

### DETAILED DESCRIPTION OF EMBODIMENTS

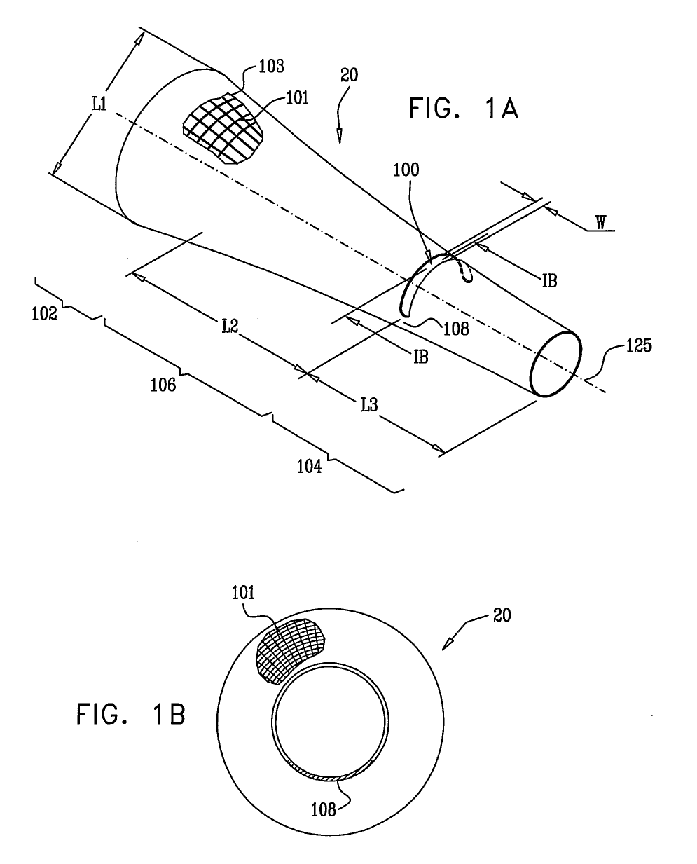

Reference is now made to FIGS. 1A and 1B, which are schematic illustrations of apparatus 20 for maintaining an extra-anatomical fistula between a first opening formed in a first body lumen and a second opening formed in a second body lumen, in accordance with an embodiment of the present invention. Typically, the first body lumen comprises a vessel, e.g., an artery, having a first pressure and the second body lumen comprises a vessel, e.g., a vein, having a second pressure that is lower than the first pressure. Apparatus 20 is typically transluminally advanced into a space, e.g., an interstitial space, defined by the first and second lumens.

Apparatus 20 comprises a substantially cylindrical endoluminal stent 101 comprising a biocompatible material, e.g., nitinol or stainless steel. Stent 101 is disposed in a contracted configuration during the advancement of apparatus 20 toward the space between the first and second lumens. Once apparatus 20 is positioned properly with respect to the first and second body lumens, apparatus 20 is passively allowed to expand, e.g., in response to retracting a sheath which surrounds apparatus 20 in order to compress apparatus 20 during advancement thereof. In some embodiments, apparatus 20 is actively expanded by expanding a balloon within a lumen defined by the stent.

In some embodiments, apparatus 20 comprises a mesh. Typically, apparatus 20 comprises a shape-memory alloy, e.g., nitinol, which enables apparatus 20 to assume a predetermined configuration once deployed within the body of the subject. In some embodiments, apparatus 20 comprises filaments which comprise a superelastic alloy. In such an embodiment, these filaments are helically wound in order to define an open weave configuration.

FIG. 1A shows a perspective side view of apparatus 20 in its expanded state. Apparatus 20 comprises a first, outwardly flared, proximal engagement portion 102, a second, distal, engagement portion 104 and a substantially cylindrical connecting portion 106 disposed intermediate the portions 102 and 104. In its expanded state, distal portion 102 is flared and is shaped to define a substantially oval transverse cross-section having a major axis that has a length L1 of between 3 mm and 6 mm. In its expanded configuration, connecting portion 106 has a diameter between 2 mm and 5 mm and a length L2 of between 3 mm and 20 mm. In its expanded configuration, engagement portion 104 is shaped to define a substantially tubular element having a length L3 of between 2 mm and 5 mm.

(In this context, in the specification and in the claims, “proximal” means closer to the opening through which apparatus described herein are originally placed into the lumens of the subject, and “distal” means further from this opening.)

Typically, apparatus 20 is used in order to generate an arteriovenous fistula in a forearm of the subject for draining blood from the radial artery into the basilic vein. Draining the blood from the artery into the vein facilitates strengthening of the radial vein for repeated insertions of hemodialysis equipment, by way of illustration and not limitation.

A 5 Fr percutaneous access is obtained in the basilic vein, roughly 5 cm distal to the wrist. Using ultrasound guidance, a suitable location for creating the arteriovenous fistula is determined. A guidewire is propagated through the access until its reached the predetermined location.

A semi-flexible, trocar having a retractable needle disposed therein is advanced over the guidewire and, under ultrasound guidance, punctures the venous wall in order to create a transvascular exit from the basilic vein. The guidewire is then interstitially advanced toward the radial artery.

The trocar is further advanced interstitially along the guidewire creating an arteriovenous channel in extravascular tissue between the basilic vein and the radial artery. The trocar is advanced until it reaches an external wall of the radial artery. The needle is then extended from within the trocar and punctures the radial artery thereby creating an opening in the radial artery.

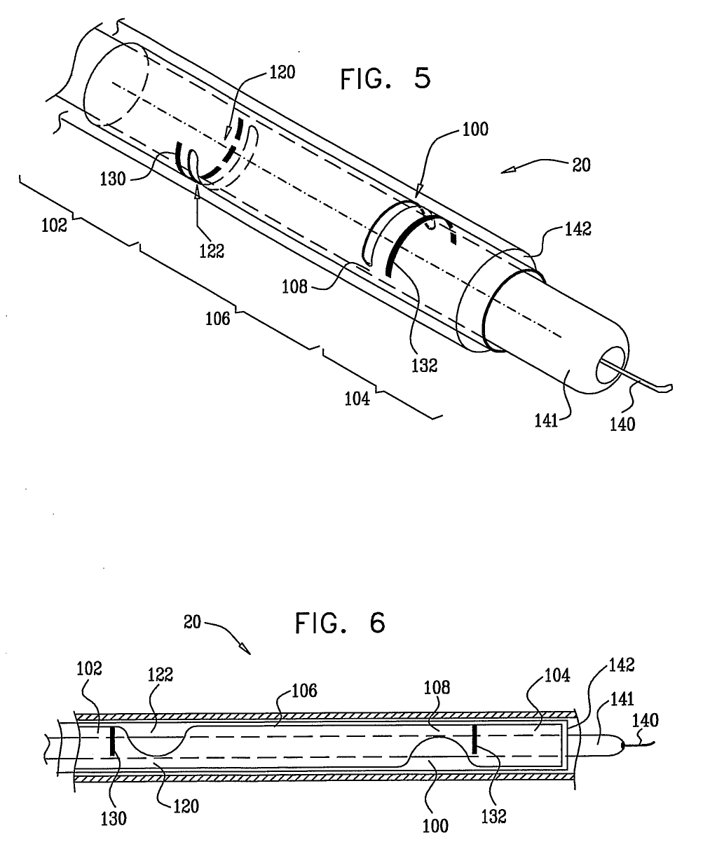

Apparatus 20 is positioned around the guidewire while being compressed within a sheath (as shown hereinbelow with reference to FIG. 5). Apparatus 20, in its compressed state is advanced along the guide wire within the basilic vein and toward the opening formed in the basilic vein. Apparatus 20 is further pushed such that distal portion 104 travels further along the guide wire through the arteriovenous channel and through the opening created in the radial artery. While apparatus 20 is pushed along the guide wire, proximal portion 102 remains disposed within the basilic vein. Apparatus is still further pushed along guide wire until (1) a distal-most end of distal portion 104 is disposed inside the lumen of the radial artery typically at a distance of at least 1.5 mm from the opening in the radial artery, and (2) a proximal-most end of proximal portion 102 is typically disposed at least 2 mm from the opening in the basilic vein.

The sheath that surrounds and compresses apparatus 20 is thereafter gradually slid off of apparatus 20 by being retracted proximally from the distal-most end of distal portion 104, thereby allowing portion 104 to expand radially until it contacts the inner wall of the radial artery. Continued sliding of sheath off of proximal portion 102 allows portion 102 to expand radially in a flared configuration from the opening formed in the vein, and thereby radially anchor itself to the inner wall of the basilic vein.

Apparatus 20 is shaped to define an aperture 100, e.g., a lateral aperture, at a junction between distal portion 104 and connecting portion 106. As shown, aperture 100 is shaped to define a slit having a width W of between 0.2 mm and 5.0 mm. Aperture 100 is shaped to define an arc subtending an angle of up to 300 degrees of the circumference at the junction between distal portion 104 and connecting portion 106. In some embodiments, the arc subtends an angle between 180 degrees and 300 degrees. Thus, distal portion 104 is connected to connecting portion 106 along a coupling junction 108 (shown in the transverse cross-section of apparatus 20 in FIG. 1B). Coupling junction 108 defines an arc of the circumference at the junction which subtends an angle between 30 degrees and 180 degrees, e.g., between 40 degrees and 80 degrees. Typically, stent 101 of apparatus 20 comprises a plurality of metal strands which are interconnected to form small openings, or windows, between the interconnected strands. It is to be noted, that, as shown in FIG. 1A, aperture 100 is larger than the size of an opening, or window, defined by the strands of stent 101.

Once apparatus 20 is deployed within the body of the subject as described hereinabove, connecting portion 106 defines a longitudinal connecting portion axis. Proximal portion 102 is configured to flare radially from the opening created within the basilic vein and away from an axis of the longitudinal connecting portion axis. Additionally, distal portion 104 bends away from connecting portion 106 via coupling junction 108. Consequently, distal portion is disposed at a non-zero angle, e.g., between 60 and 90 degrees, with respect to the longitudinal connecting portion axis of connecting portion 106, and is aligned in parallel with a longitudinal axis defined by the radial artery.

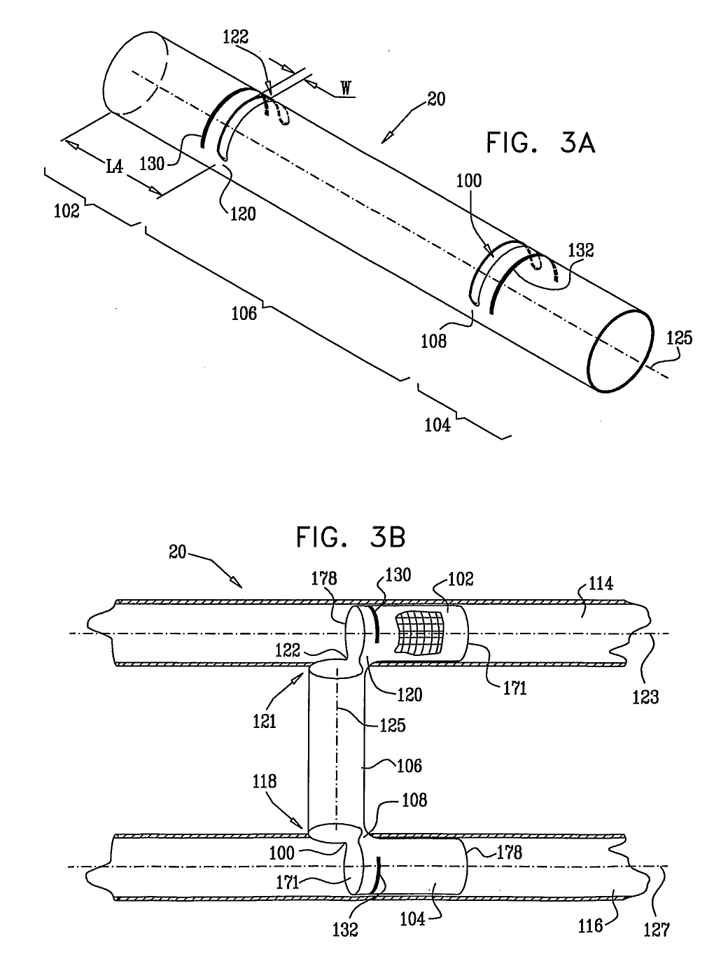

Aperture 100 enables apparatus 20 to be deployed in such a way that facilitates unobstructed flow of blood within the artery. Since distal portion 104 is coupled to connection portion 106, via coupling junction 108 defining an arc of 30-180 degrees, e.g., 40-80 degrees, the deployed configuration of distal portion 104 with respect to connecting portion 106 minimizes the formation of fibrosis within a transverse plane that is substantially perpendicular to the flow of blood within the radial artery. As shown in its deployed configuration in FIG. 3B, distal portion 104 provides an unobstructed channel for blood to flow without being blocked in its path by any portion of apparatus 20. That is, because aperture 100 facilitates the bending of portion 104 only along junction 108, there is no portion of apparatus 20 which is disposed substantially perpendicularly with respect to the direction of blood flow, but rather portion 104 is disposed within the body lumen such that the wall of portion 104 contacts the wall of the body lumen and the longitudinal axis of the lumen of portion 104 is in parallel with respect to the direction of blood flow.

Typically, following implantation, extravascular interstitial tissue surrounding connecting portion 106 initiates neovascularization surrounding portion 106. The interstitial tissue creates the vaso-vasorum and initiates intimization and eventually vascularization of the arteriovenous channel in which connecting portion 106 is disposed. Typically, a biodegradable film 103 surrounds stent 101 and is configured to degrade within less than 90 days following implantation of apparatus 20 within the body of the subject. During the vascularization of the arteriovenous channel, film 103 is configured to minimize leaking of blood as it is passes through connecting portion 106. The newly vascularized tissue then functionally replaces the film and functions to keep blood within and maintain blood flow through the fistula apparatus 20.

It is to be noted that apparatus described herein for creating a fistula may be used independently of or in combination with film 103.

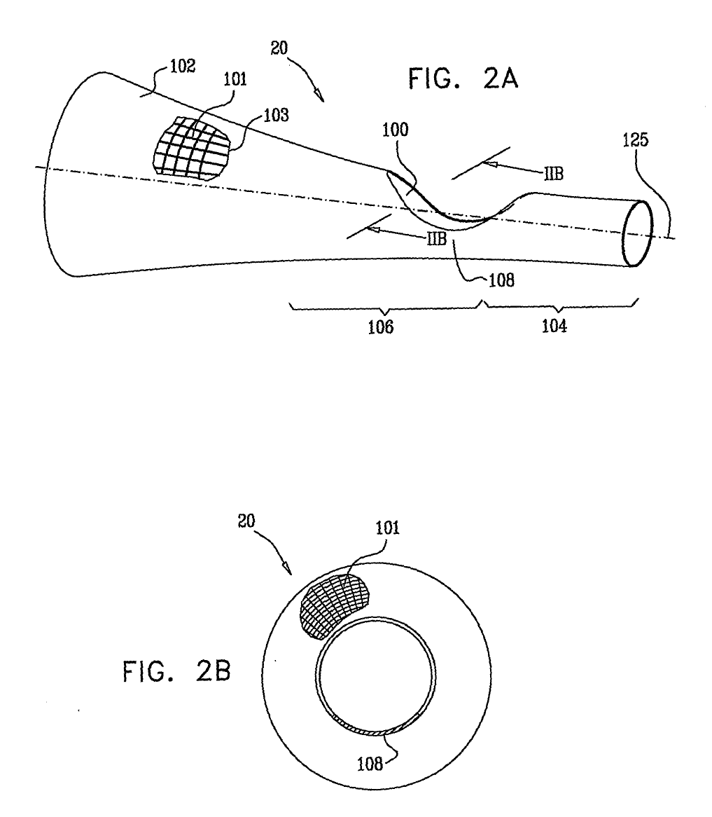

Reference is now made to FIGS. 2A and 2B which are schematic illustration of apparatus 20 as described hereinabove with reference to FIGS. 1A and 1B, with the exception that aperture 100 is shaped to define a partially ellipsoidal aperture having an elliptical cross-section along the longitudinal axis of apparatus 20, in accordance with an embodiment of the present invention. Aperture 100 is shaped to define a length L5 of its major axis between 2 mm and 5 mm.

Reference is now made to FIG. 3A, which is a schematic illustration of apparatus 20 as described hereinabove with reference to FIG. 1A, with the exception that apparatus 20 is shaped to define a second aperture 122, in accordance with an embodiment of the present invention. Apparatus 20 is shaped to define aperture 122, e.g., a lateral aperture, at a junction between proximal portion 102 and connecting portion 106. As shown, aperture 122 is shaped to define a slit having a width W of between 0.2 mm and 5.0 mm. Aperture 100 is shaped to define an arc subtending an angle of up to 300 degrees of the circumference at the junction between proximal portion 102 and connecting portion 106. In some embodiments the arc subtends an angle between 180 degrees and 300 degrees. Thus, proximal portion 102 is connected to connecting portion 106 along a coupling junction 120. Coupling junction 120 defines an arc of the circumference at the junction which subtends an angle between 30 degrees and 180 degrees, e.g., 80 degrees.

Reference is now made to FIGS. 3A and 3B. FIG. 3B shows apparatus 20 as described in FIG. 3A in an expanded state, creating an extra-anatomical fistula between an opening 121 of first lumen 114 and a second opening 118 of second lumen 116, in accordance with an embodiment of the present invention. In an expanded state, proximal engagement portion 102 is shaped to define a substantially tubular element having a length L4 (shown in FIG. 3A) between 2 mm and 5 mm.

For embodiments in which first body lumen 114 comprises an artery and second body lumen 116 comprises a vein, connecting portion 106 is disposed interstitially between first body lumen 114 and the second body lumen 116 and defines a substantially longitudinal connecting portion axis 125 thereof. Proximal engagement portion 102 is disposed within first body lumen 114, and defines a first engagement portion axis 123 thereof which aligns in parallel with a longitudinal axis of lumen 114. Similarly, distal engagement portion 104 is disposed within second body lumen 116, and defines a second engagement portion axis 127 thereof which aligns in parallel with a longitudinal axis of lumen 116. Once deployed, connecting portion axis 125 is configured to align at a non-zero angle (e.g., substantially perpendicularly, as shown) with respect to axes 123 and 127.

Reference is now made to FIGS. 3A and 3B. Junctions 108 and 120 are disposed along the same lateral surface of apparatus 20 (FIG. 3A). Once deployed, distal portion 104 and proximal portion 102 bend away from connecting portion 106 via coupling junction 108 and 120, respectively. Portions 102 and 104 bend away from connecting portion 106 such that they are both disposed within the same side of axis 125. Distal portion 104 and proximal portion 102 are disposed substantially perpendicularly, e.g., between 60 and 90 degrees, with respect to longitudinal connecting portion axis 125 of connecting portion 106.

Apertures 100 and 122 enable apparatus 20 to be deployed in such a way that facilitates unobstructed flow of fluid within lumens 116 and 114, respectively, as described hereinabove with respect to aperture 100. Once deployed, engagement portions 102 and 104 of apparatus 20 define (1) respective channels having a diameter substantially equal to the respective diameters of first and second body lumens 114 and 116, and (2) proximal and distal openings 171 and 178 having a diameter substantially equal to the respective diameters of first and second body lumens 114 and 116. Openings 171 and 178 are shown in their expanded states as being substantially circular in cross-section and having a diameter which conforms to the diameter of lumen 114. Openings 171 and 178 minimize the formation of fibrosis within respective transverse planes of the first and second portions that are substantially perpendicular to the flow of blood within first and second body lumens 114 and 116.

It is to be noted that proximal engagement portion 102 and distal engagement portion 104 are shown as comprising one tubular element by way of illustration and not limitation. For example, proximal engagement portion 102 may comprise a first tubular element and a second tubular element.

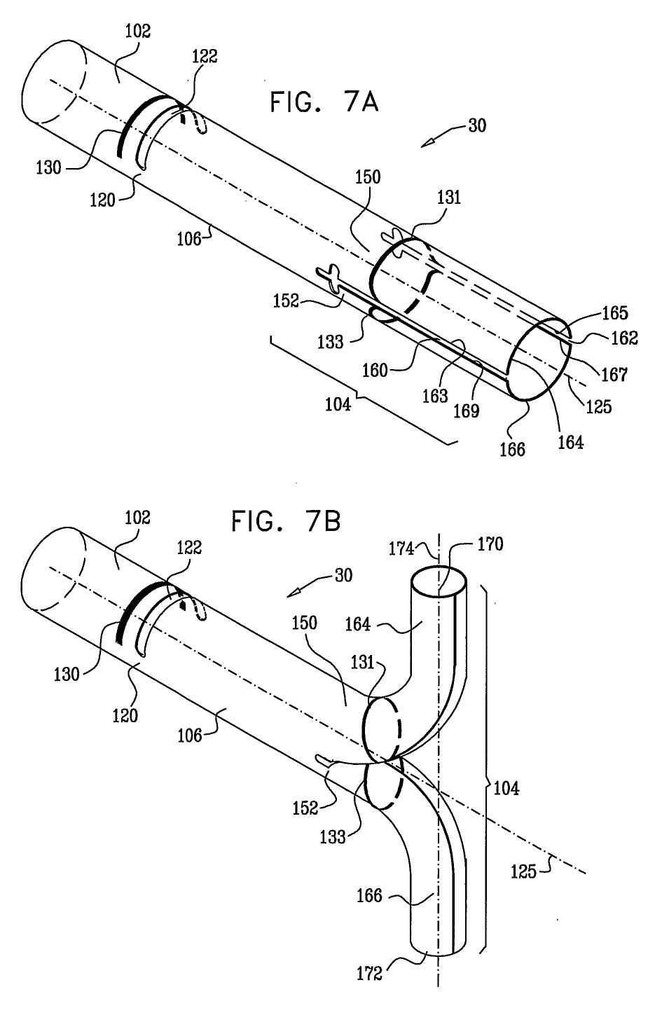

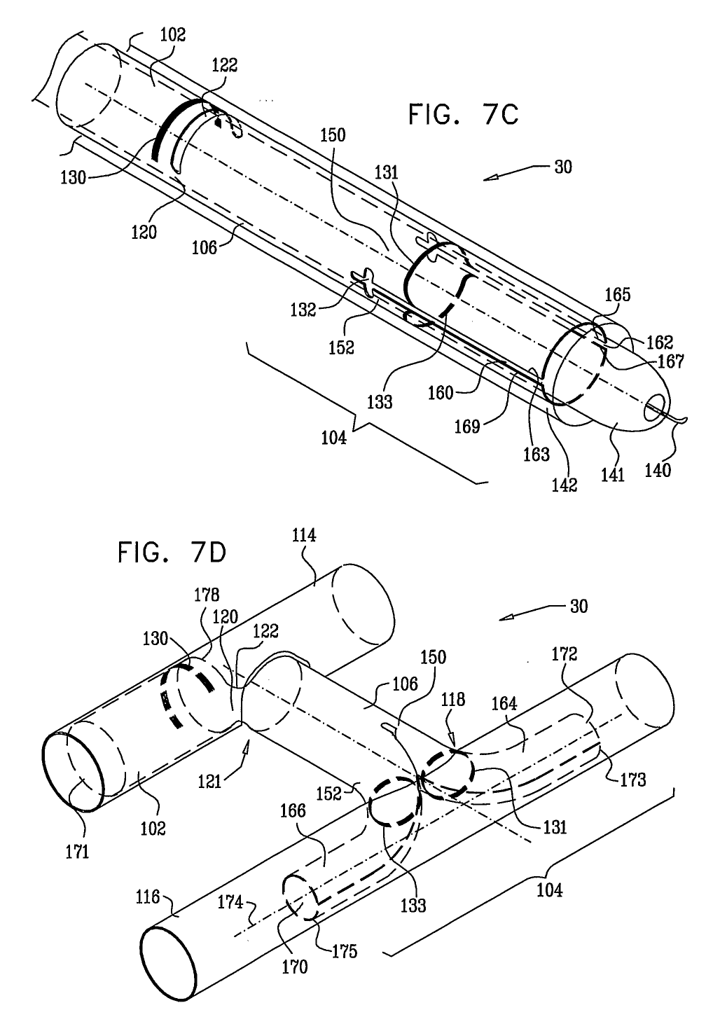

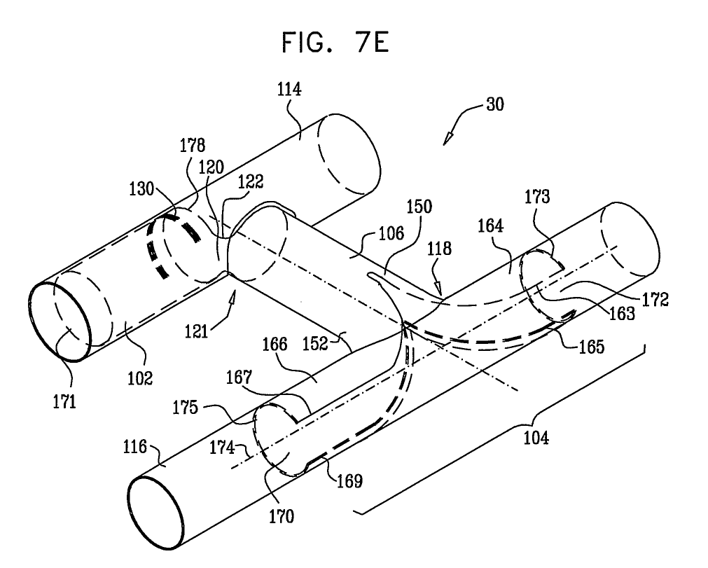

In some embodiments, the first and second tubular elements of portion 102 comprise a first stent and a second stent, respectively as shown in FIGS. 7A-E. The first and second tubular elements each are coupled to connecting portion 106 along respective first and second arcs of the circumference of the proximal end of connecting portion 106. The first and second arc each subtend respective first and second angles that are each less than 180 degrees, e.g., less than 90 degrees. In some embodiments, the sum of the first and second angles is less than 180 degrees, e.g., less than 90 degrees. Once deployed within first body lumen 114, the first tubular element is disposed proximally adjacently to opening 121, i.e., in proximity to a first side of opening 121, and the second tubular element is disposed distally adjacently to opening 121, i.e., in proximity to a second side of opening 121. Once deployed, the first and second tubular elements are each aligned substantially in parallel with a longitudinal axis of body lumen 114.

Once deployed, the first and second tubular elements of first engagement portion 102 are disposed substantially perpendicularly, e.g., between 30 degrees and 90 degrees, with respect to longitudinal connecting portion axis 125 of connecting portion 106. The first and second elements of portion 102 are configured to facilitate substantially unobstructed flow of fluid within lumen 114, as described hereinabove.

It is to be noted that distal engagement portion 102 may also comprise first and second tubular elements which are similar to the first and second tubular elements of portion 102 as described hereinabove.

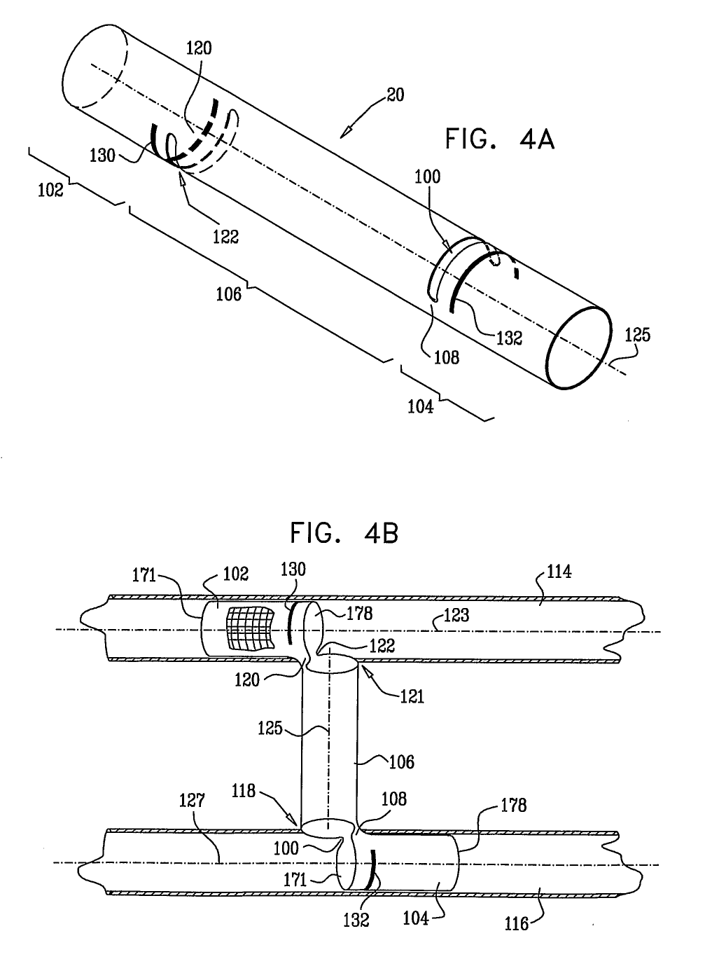

Reference is now made to FIGS. 4A and 4B, which are schematic illustrations of apparatus 20 as described hereinabove with reference to FIGS. 3A and 3B, with the exception that junctions 108 and 120 are disposed along opposing lateral surfaces of apparatus 20, in accordance with an embodiment of the present invention. Once deployed, portions 102 and 104 bend away from connecting portion 106 such that they are both disposed within opposing sides of axis 125.

FIG. 5 shows apparatus 30 described hereinabove with reference to FIG. 3A in a radially collapsed configuration, in accordance with an embodiment of the present invention. As shown, distal engagement portion 104 comprises a radiopaque marker 132 which is disposed distally to aperture 100. Typically, marker 132 is shaped to define a partial circle. During the transluminal advancement of apparatus 20, the orientation of the partial circle of marker 132 indicates the directionality, orientation, and positioning of distal engagement portion 104 within the first body lumen. Marker 132 is configured to provide an indication of the orientation of portion 104 with respect to connecting portion 106. Additionally, marker 132 indicates the position of aperture 100. Marker 132 helps indicate this position of aperture 100 during the exposure of aperture 100 as the sheath (described hereinabove) surrounding apparatus 20 is slid proximally therealong.

Similarly, proximal engagement portion 102 comprises a radiopaque marker 130 which is disposed proximally to aperture 122. Typically, marker 130 is shaped to define a partial circle. During the transluminal advancement of apparatus 20, the orientation of the partial circle of marker 130 indicates the directionality and positioning of proximal engagement portion 102 within the first body lumen. Marker 130 is configured to provide an indication of the orientation of portion 102 with respect to connecting portion 106. Additionally, marker 130 indicates the position of aperture 122. Marker 130 helps indicate this position of aperture 122 during the exposure of aperture 122 as the sheath (described hereinabove) surrounding apparatus 20 is slid proximally therealong.

As shown, apparatus 20 is compressed between a sheath 142 and a catheter 141. Typically, catheter 141 is slid along a guidewire 140 (described hereinabove) toward the location designated for the creation of the fistula. In some embodiments, guidewire 140 comprises nitinol. Following the creation of the fistula, guidewire 140, catheter 141, and sheath 142 are extracted from within the subject's vasculature.

FIG. 6 shows apparatus 20 as described hereinabove with reference to FIG. 5, with the exception that apertures 100 and 122 are shaped to define the partially ellipsoidal apertures, in accordance with an embodiment of the present invention. Apertures 100 and 120 define respective coupling junctions 108 and 120 wherein each junction defines an arc subtending an angle less than 180 degrees, e.g., less than 80 degrees.

Reference is now made to FIGS. 7A-D, which are schematic illustrations of an apparatus 30 for creating a fistula having a lateral aperture 122 and first and second longitudinal apertures 160 and 162, in accordance with an embodiment of the present invention. FIG. 7A shows apparatus 30 as described herein with respect to apparatus 20, with the exception that distal engagement portion 104 is shaped to define apertures, e.g., slits or elliptical apertures, 160 and 162.

As shown in FIG. 7B, once deployed, first longitudinal slit 160 and second longitudinal slit 162 facilitate the formation of a first curved, semi-cylindrical portion 164 and a second curved, semi-cylindrical portion 166. Portions 164 and 166 are coupled to connecting portion 106 along respective coupling junctions 150 and 152. As shown, apertures 160 and 162 define slits. In such an embodiment, coupling junctions 150 and 150 define respective arcs which each subtend substantially 180 degree angles. Alternatively, apertures 160 and 162 define elliptical apertures. In such an embodiment, coupling junctions 150 and 152 define respective arcs each subtending angles between 60 degrees and 180 degrees, e.g., between 90 degrees and 120 degrees or between 40 degrees and 90 degrees. In some embodiments, the sum of the angles is less than 180 degrees, e.g., between 90 and 120 degrees or between 40 degrees and 90 degrees.

Typically, apparatus 30 comprises a shape-memory alloy, nitinol, which is configured to assume the shape illustrated in FIG. 7B. Once allowed to expand, portions 164 and 166 bend radially away from connecting portion 106 along junctions 150 and 152, respectively. Consequently, distal portion 104 is disposed substantially perpendicularly, e.g., between 60 and 90 degrees, with respect to longitudinal connecting portion axis 125 of connecting portion 106. Concurrently with the bending, lateral surfaces 163 and 165 assume their predetermined shape by curling toward each other, thereby defining a first cylinder comprising first curved portion 164. Similarly, lateral surfaces 167 and 169 assume their predetermined shape by curling toward each other, thereby defining a second cylinder comprising second curved portion 166. The first and second cylinders define respective first and second lumens 170 and 172. Lumens 170 and 172 are disposed along a generally coinciding longitudinal axis 174.

FIG. 7C schematically depicts apparatus 30 described in FIGS. 7A and 7B, disposed in a radially collapsed configuration and mounted over a conventional stent delivery guidewire 140. Additionally, apparatus 30 is compressed between sheath 142 and catheter 141. Typically, catheter 141 is advanced around guidewire 140 and toward the first opening formed in the first body lumen. Distal portion 104 of apparatus 30 is pushed along guide wire 140 and through the first opening of the first body lumen 114, through the channel between the first and second body lumens, through the second opening made in the second body lumen 116 until (1) a distal end of distal portion 104 is disposed inside the lumen of second lumen 116, while (2) proximal portion 102 remains disposed within the first body lumen 114 adjacently to the first opening formed therein.

Sheath 142 is thereafter gradually slid proximally from the distal-most end of portion 104 thereby allowing surfaces 164 and 166 to assume their respective radially expanded, cylindrical configurations, as shown in FIG. 7D. Sheath 142 is further slid proximally until proximal engagement portion 102 is allowed to expand within first body lumen 114, in a manner as described hereinabove with reference to FIG. 1A, with respect to the expansion of distal engagement portion 104. Following deployment, guidewire 140, catheter 141, and sheath 142 are extracted from within the body of the subject.

As shown, distal engagement portion 104 comprises first and second radiopaque markers 131 and 133 which are coupled to portions 164 and 166, respectively. Typically, markers 131 and 133 are shaped to define partial circles which are disposed at a tilt with respect to axis 125 of apparatus 30. During the transluminal advancement of apparatus 30, the orientation of the partial circles of markers 131 and 133 indicate the directionality, orientation, and positioning of distal engagement portion 104 with respect to connecting portion 106. Similarly, proximal engagement portion 102 comprises radiopaque marker 130 which is disposed proximally to aperture 122. Typically, marker 130 is shaped to define a partial circle. During the transluminal advancement of apparatus 30, the orientation of the partial circle of marker 130 indicates the directionality and positioning of proximal engagement portion 102 within the first body lumen.

Once deployed (FIG. 7D), engagement portion 104 of apparatus 30 defines (1) respective lumens 170 and 172 having respective diameters that are smaller than the diameter of body lumen 116, and (2) proximal and distal openings 173 and 175 having respective diameters that are smaller than the diameter of second body lumen 116. Openings 173 and 175 minimize the formation of fibrosis within respective transverse planes of the first and second portions 164 and 166 that are substantially perpendicular to the flow of blood within second body lumen 116, as described hereinabove with respect to openings 171 and 178 with reference to FIGS. 3A-B. Once distal engagement portion 104 of apparatus 30 is deployed within lumen 116, radiopaque markers 131 and 133 are aligned substantially parallel with respect to axis 174 of the first and second cylinders.

Reference is now made to FIG. 7E, which is a schematic illustration of apparatus 30 as described hereinabove with reference to FIG. 7D, with the exception that curved portions 164 and 166 engage an inner wall of lumen 116, in accordance with an embodiment of the present invention. Once deployed (FIG. 7D), engagement portion 104 of apparatus 30 defines (1) respective lumens 170 and 172 having respective diameters that are substantially less than or equal to a diameter of body lumen 116, and (2) proximal and distal openings 173 and 175 having respective diameters that are substantially equal to the diameter of second body lumen 116. As described hereinabove, openings 171 and 178 minimize the formation of fibrosis within respective transverse planes of the first and second portions 164 and 166 that are substantially perpendicular to the flow of blood within second body lumen 116.

It is to be noted that distal engagement portion 104 comprises radiopaque markers 131 and 133 as described hereinabove. Markers 131 and 133 are not shown in FIG. 7E for clarity of illustration.

In some embodiments, a first guidewire is used to facilitate the deployment of first cylindrical surface 164, and a second guidewire is used to facilitate the deployment of second cylindrical surface 166.

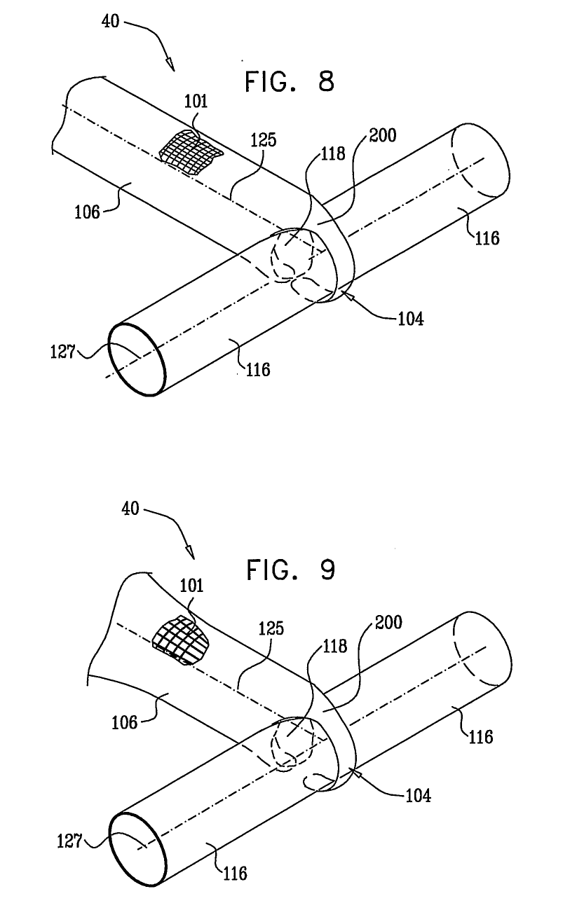

FIG. 8 is a schematic illustration of an apparatus 40 for creating a fistula between first and second body lumens, the apparatus comprising at least one projection 200, in accordance with an embodiment of the present invention. Typically, projection 200 comprises an elastic shape-memory alloy, e.g., nitinol, configured to assume a predetermined shape (e.g., cylindrical, as shown) once apparatus 40 is allowed to expand within the body of the subject. Typically, projection 200 surrounds body lumen 116 adjacently to opening 118 formed therein. Such configuration facilitates fluid communication between lumen 116 and connecting portion 106. Fluid drains from lumen 116, through connecting portion 106 and into body lumen 114 (not shown).

In some embodiments, proximal engagement portion 102, which is configured to engage an internal wall of first body lumen 114, comprises a substantially tubular element as described hereinabove with reference to FIG. 3A. In some embodiments, proximal engagement portion 102 comprises first and second tubular elements and each element is coupled to connecting portion 106 along a respective coupling junction (as described hereinabove with reference to FIGS. 7A-E. In such an embodiment, the coupling junctions define respective arcs subtending respective angles each between 60 degrees and 180 degrees, e.g., between 90 degrees and 120 degrees or between 40 degrees and 90 degrees. In some embodiments the sum of the angles of the coupling junctions is less than 180 degrees, e.g., between 90 degrees and 120 degrees or between 40 degrees and 90 degrees. In some embodiments, proximal engagement portion 102 is shaped to define a flared configuration, as described hereinabove with reference to FIG. 1A.

Projection 200 a of second engagement portion 104 engages an external wall of body lumen 116. Projection 200 is configured to be wrapped around at least a portion of a circumference of lumen 116 (360 degrees as shown).

The implantation of apparatus 40 is similar to the implantation of apparatus 20 as described hereinabove with reference to FIG. 1A. A 5 Fr percutaneous access is obtained in a first body lumen. Using ultrasound guidance, a suitable location for creating the fistula is determined. A guidewire is advanced through the access until is reaches the predetermined location at the wall of the first body lumen. A semi-flexible, trocar having a retractable needle disposed therein is advanced over the guidewire and, under ultrasound guidance, punctures the wall of the first vessel in order to create a transluminal exit from the first lumen. The guidewire is then advanced toward the second body lumen.

The trocar is further advanced along the guidewire until it reaches an external wall of the second body lumen. As the trocar is advanced, it creates a channel intermediate the first and second body lumens. The needle is extended from within the trocar and punctures the second body lumen thereby creating an opening in the second body lumen.

Apparatus 40 is compressed within a sheath and is fed over the guide wire and is advanced therealong and toward the opening formed in the first body lumen. Distal portion 104 of apparatus 40 is pushed until (1) projection 200 of distal portion 104 is abuts the external wall of the second body lumen, and (2) a proximal-most end of proximal portion 102 is disposed at least 2 mm from the opening in the first body lumen.

The sheath is thereafter gradually slid off of apparatus 40 by being retracted proximally from projection 200 of distal portion 104, thereby allowing projection 200 to expand, wrap around and surround at least in part the external wall of lumen 116. Projection 200 typically surrounds the external wall in proximity to the opening formed therein.

Reference is now made to FIG. 9, which is a schematic illustration of apparatus 40 as described hereinabove with reference to FIG. 8, with the exception that projection 200 partially surrounds the external wall of lumen 116, in accordance with an embodiment of the present invention. As shown, projection 200 surrounds at least 270 degrees of a circumference of the external wall of lumen 116.

FIG. 10 shows apparatus 40 as described hereinabove with reference to FIG. 9, with the exception that distal engagement portion 104 comprises a bifurcated projection 206; in accordance with an embodiment of the present invention. Bifurcated projection 206 is shaped to define a first subprojection 202 and a second subprojection 204. First and second subprojections 202 and 204 are spaced apart from each other at an external surface of lumen 116, thereby increasing stability of the coupling between projection 206 and lumen 116. Bifurcated projection 206 wraps around the external surface of lumen 116 at least 290 degrees. Subprojection 204 is configured to be disposed proximally adjacently to second opening 118, i.e., disposed in proximity to a first side of opening 118. Subprojection 202 is configured to be disposed distally adjacently to second opening 118, i.e., disposed in proximity to a second side of opening 118.

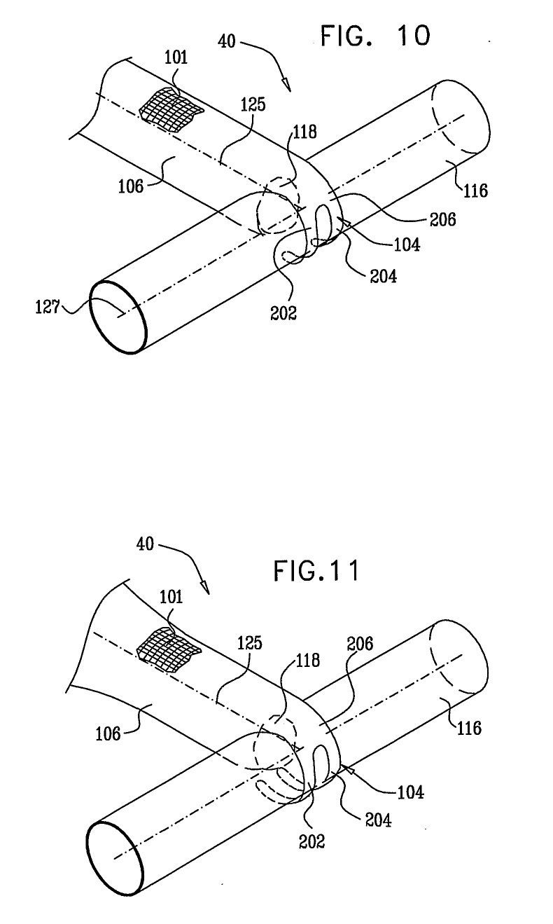

FIG. 11 is a schematic illustration of apparatus 40 as described hereinabove with respect to FIG. 10, with the exception that projection 206 surrounds substantially 360 degrees of the external wall of lumen 116, in accordance with an embodiment of the present invention.

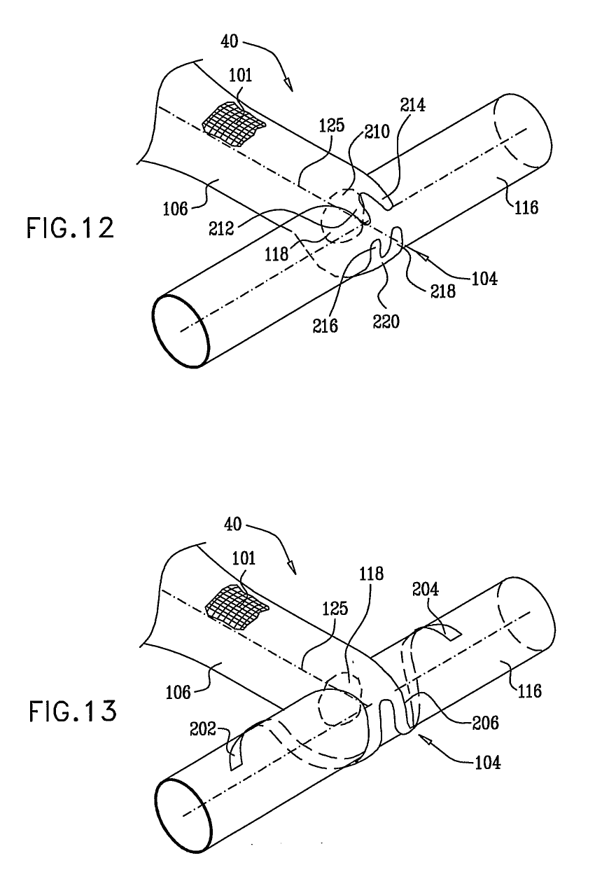

Reference is now made to FIG. 12, which is a schematic illustration of distal portion 104 comprising two bifurcated projections 210 and 220 which surround the external wall of lumen 116 in proximity to an opening formed therein, in accordance with an embodiment of the present invention. Bifurcated projection 210 surrounds at least a portion, e.g., 150 degrees, of an upper lateral surface of lumen 116 and is shaped to define a first upper subprojection 212 and a second upper subprojection 214. Bifurcated projection 220 surrounds at least a portion, e.g., 150 degrees, of a lower lateral surface of lumen 116 and is shaped to define a first lower subprojection 216 and a second lower subprojection 218. The relative spatial configuration of upper bifurcated projection 210 and lower bifurcated projection 220 is such that projections 210 and 220 are disposed with respect to each other so as to resemble a shape of a cylinder, jointly spanning approximately 300 degrees of a circumference of lumen 116 in the vicinity of opening 118 formed therein.

FIG. 13 is a schematic illustration of apparatus 40 as described hereinabove with respect to FIG. 11, with the exception that projection 206 helically surrounds the external wall of lumen 116, in accordance with an embodiment of the present invention. As shown by way of illustration and not limitation, bifurcated subprojections 202 and 204 expand around lumen 116 such that subprojections 202 and 204 form respective spirals. Each spiral spans approximately 540 degrees, i.e., one complete 360 degree turn and an additional 180 degrees. Subprojection 204 is configured to be surround lumen 116 by wrapping itself proximally adjacently to second opening 118, i.e., in proximity to a first side of opening 118. Subprojection 202 is configured to surround lumen 116 by wrapping itself distally adjacently to second opening 118, i.e., in proximity to a second side of opening 118.

Reference is now made to FIGS. 1A-B, 2A-B, 3A-B; 4A-B, 5-6, and 7A-E. It is to be noted that the apertures 100 described herein each have a characteristic diameter, that is typically between one half and two times the diameter of the stent in an expanded configuration thereof. For embodiments in which the aperture is shaped to define a circle, the characteristic diameter of the circle is the diameter of the circle. For embodiments in which the aperture is shaped to define an ellipse, the characteristic diameter of the ellipse is the average length of the major axis and the minor axis. Other aperture shapes have corresponding characteristic diameters which can be attributed to them.

It is to be noted that the scope of the present invention includes surgical placement of apparatus described herein. It is to be further noted that engagement portions 102 and 104 described herein are shown by way of illustration and not limitation and that portions 102 and 104 may assume any configuration suitable for engaging an internal wall and/or an external wall of a body lumen of the subject. For example, engagement portions 102 and/or 104 may comprise substantially tubular elements. In some embodiments, engagement portions 102 and/or 104 may be shaped to define a flared configuration described hereinabove. In some embodiments, engagement portions 102 and/or 104 may comprise projections 200 or 206 described hereinabove.

For some applications, techniques described herein are practiced in combination with techniques described in one or more of the patent references above, or the references cited in the Background section or Cross-references section of the present patent application, which are incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-175. (canceled)

176. An apparatus for creating a connection between a first body lumen and a second body lumen, the apparatus comprising:
at least one first engagement portion configured to engage the inner wall of the first body lumen; at least one second engagement portion configured to engage the inner wall of the second body lumen; a substantially cylindrical connecting portion connecting said first engagement portion to said second engagement portion, the connecting portion being configured to define a substantially longitudinal flow channel therethrough for passage of fluid from the inner space of the first body lumen, through the flow channel, and into the inner space of the second body lumen, such that the flow of fluid within the body lumen in contact with the first and/or second engagement portions is substantially unobstructed.

177. The apparatus of claim 176, comprising one or more apertures cut into said cylindrical portion, said apertures configured to define flow channel junctions between said respective engagement portions and said connecting portion.

178. The apparatus of claim 176, wherein said connecting portion includes one or more elements selected from the group consisting of a self-expanding stent, a shape memory alloy, a mesh, and a superelastic alloy, configured to assume a predetermined shape.

179. The apparatus of claim 176, wherein at least one of said engagement portions is shaped to define a flared configuration.

180. The apparatus of claim 176, wherein at least one of said engagement portions comprises an expandable tubular element.

181. The apparatus of claim 176, wherein at least one of said engagement portions comprises a radiopaque marker configured to indicate an orientation of said engagement portion with respect to said connecting portion.

182. The apparatus of claim 176, wherein said second engagement portion is configured to be disposed substantially perpendicularly with respect to the axis of said connecting portion.

183. The apparatus of claim 176, said first engagement portion is configured to be disposed within the first body lumen in proximity to a first side of the first opening formed therein, said second engagement portion is configured to be disposed within the first body lumen in proximity to a second side of the first opening formed therein, and the first and second engagement portions are each aligned substantially in parallel with a longitudinal axis of the first and second body lumen.

184. The apparatus of claim 176, wherein said longitudinal flow channel is at least partially surrounded by a biodegradable film, said biodegradable film configured to define a fluid-impervious lumen.

185. The apparatus of claim 176, further comprising at least one projection portion coupled to said second engagement portion, said projection portion configured to surround at least a portion of an external surface of the wall of said second body lumen.

186. The apparatus of claim 185, wherein said projection portion comprises a shape-memory alloy configured to assume a predetermined shape once in expanded form.

187. The apparatus according to claim 185, wherein said projection portion comprises a bifurcated projection being bifurcated to a first subprojection and a second subprojection, and wherein said first subprojection is configured to surround at least in part a lateral surface of the second body lumen proximally adjacent to the second opening, and the second subprojection is configured to surround at least in part the lateral surface, distally adjacent to the second opening.

188. A method for transluminally creating a fistula between a first body lumen of a subject and a second body lumen of the subject, the method comprising:
entering a catheter into the first body lumen; creating a first opening in the first body lumen, using a tissue penetrating member; creating a second opening in the second body lumen, using said tissue penetrating member; advancing into said first opening at least a portion of a substantially tubular element in a compressed state; advancing into said second opening at least a portion of said tubular element in a compressed state;
engaging the first lumen with at least a portion of said proximal portion of said tubular element, such that a substantially unobstructed flow of fluid within the first lumen is maintained; engaging the second lumen with said distal portion of said tubular element, such that a substantially unobstructed flow of fluid within the second lumen is maintained; and expanding said tubular element to define a substantially longitudinal flow channel therethrough for passage of fluid from the first body lumen through to the second body lumen.

189. The method of claim 188 wherein said engaging of said first and said second lumen comprises anchoring said distal and proximal portions respectively of said tubular element to at least a portion of the circumference of the walls of the first and the second lumen.

190. The method of claim 188, wherein said engaging actions are executed by expanding a shape-memory alloy.

191. The method of claim 188, comprising bending said tubular element by one or more integrated apertures, such that said bending forms a substantially longitudinal flow channel junction.

192. The method of claim 188, comprising widening of said longitudinal flow channel to create an arteriovenous channel.

193. The method of claim 188, comprising bending said tubular element away from said longitudinal flow channel, along an axis substantially perpendicular to said longitudinal flow channel.

194. The method of claim 188, comprising advancing a tubular element surrounded by a biodegradable film.

195. The method of claim 188, comprising expanding at least one projection portion coupled to said distal portion of said tubular element, to surround at least a portion of an external surface of the wall of the second body lumen.

196. A stent, comprising:
a proximal portion, including a first engagement portion configured to engage the arc of the circumference of a first body lumen, such that a substantially unobstructed flow of fluid within the first body lumen is maintained;
a distal portion, including a second engagement portion configured to engage the arc of the circumference of a second body lumen, such that a substantially unobstructed flow of fluid within the second body lumen is maintained; and
a connecting portion disposed intermediate said proximal and distal portions, said connecting portion defining a substantially longitudinal flow channel between a first flow junction formed at a first aperture in the stent, and a second flow junction formed at a second aperture in the stent.
